(19) United States
(12) Patent Application Publication
(10) Pub. No.: US 2024/0285861 A1
(43) Pub. Date: Aug. 29, 2024

(54) SHAPE-CHANGEABLE SYRINGE PACKAGE SYSTEM

(71) Applicant: Becton, Dickinson and Company, Franklin Lakes, NJ (US)

(72) Inventors: Shashwat Jain, Indore (IN); Vineeth Acharya, Udupi (IN); Senthilkumar Rakkiyappan, Coimbatore (IN); Praveen Nalawade, Belgaum (IN)

(73) Assignee: Becton, Dickinson and Company, Franklin Lakes, NJ (US)

(21) Appl. No.: 18/113,966

(22) Filed: Feb. 24, 2023

(51) Int. Cl. A61M 5/31; A61M 5/00

(52) U.S. Cl. CPC A61M 5/31; A61M 5/002

(57) ABSTRACT

A syringe and syringe package system comprises a syringe barrel having an open proximal end and a distal end and a syringe stopper placed in the open proximal end of the syringe barrel. The system includes a shape-changeable package, the syringe barrel and the syringe stopper contained within the shape-changeable package, wherein the shape-changeable package comprises a web of material sized and arranged to contain the syringe barrel and the syringe stopper and configured to be converted to a plunger rod that engages the syringe stopper and is translatable within the open proximal end of the barrel.

### TECHNICAL FIELD

The present disclosure generally relates to medical syringes and packages, and in particular, syringes packaged in a shape-changeable package.

### BACKGROUND

Disposable medical devices and medical device packaging made from plastics are a significant source of landfill waste. Increasing use of plastics over the past decades has resulted in increased impact on landfill capacity and the depletion of fossil fuel-based resources. Syringes, include a syringe barrel and a plunger and stopper are typically packaged in a blister package or clamshell blister package made from plastic. Plastic medical devices and plastic packaging used for medical devices contributes to increased levels of environmental pollution, associated carbon footprint and other environmental impacts. While medical waste may be incinerated, incineration can create a potential air pollution issue.

Recycling of some medical device products such as syringes and packaging may be viable, but not all medical facilities such as hospitals, urgent care facilities, medical offices and long-term care facilities may not be able to or willing to comply with recycling of used medical devices. Issues of cost and convenience in complying with recycling of used medical devices may prevent widespread adoption of recycling of used medical devices and packaging. In addition, supply chains must be established for collection of the used devices and packaging, shipping, shredding, disinfection of the used medical devices and reuse of recycled plastic.

It would be desirable to provide alternative solutions to the problem of waste generated by the used of medical devices including syringes and associated packaging.

### SUMMARY

In a first embodiment a syringe and syringe package system comprises a syringe barrel having an open proximal end and a distal end; a syringe stopper placed in the open proximal end of the syringe barrel; and a shape-changeable package, the syringe barrel and the syringe stopper contained within the shape-changeable package, wherein the shape-changeable package comprises a web of material sized and arranged to contain the syringe barrel and the syringe stopper and configured to be converted to a plunger rod that engages the syringe stopper and is translatable within the open proximal end of the barrel.

In a second embodiment, syringe and syringe package system comprises a syringe barrel having an open proximal end and a distal end; a syringe stopper placed in the open proximal end of the syringe barrel; and a shape-changeable package, the syringe barrel and the syringe stopper contained within the shape-changeable package, wherein the shape-changeable package comprises a rolled web of biodegradable material sized and arranged to contain the syringe barrel and the syringe stopper and configured to be converted to a plunger rod that engages the syringe stopper and is translatable within the open proximal end of the syringe barrel.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a perspective view of a syringe and a shape-changeable package according to a first embodiment;

FIG. 2 is a side view of the syringe and a shape-changeable package shown in FIG. 1 with the syringe partially removed from the shape-changeable package;

FIG. 3 is a side view of the syringe and a shape-changeable package shown in FIG. 1 with the syringe fully removed from the shape-changeable package;

FIG. 4A is front left perspective view of the shape-changeable package shown in FIG. 1;

FIG. 4B is front right perspective view of the shape-changeable package shown in FIG. 1;

FIG. 5 is a partial perspective view of the shape-changeable package configured as a plunger rod and prior to assembly with a stopper

FIG. 6 is a partial cutaway view side view of a syringe stopper and a plunger rod inserted into the syringe stopper;

FIG. 7 is a front perspective view of a second embodiment of a syringe and a shape-changeable package;

FIG. 8A is a front perspective view of the shape changeable package shown in FIG. 7;

FIG. 8B is a front perspective view of the shape changeable package shown in FIG. 8A configured as a plunger rod;

FIG. 9A is a perspective view of the shape changeable package configured as a plunger rod shown in FIG. 8B being inserted into a stopper of a syringe;

FIG. 9B shows a detailed view of the shape changeable package configured as a plunger rod shown in FIG. 9A being inserted into a stopper of a syringe;

FIG. 10 shows a side view of the shape changeable package shown in FIG. 7

FIG. 11 is a perspective view of a third embodiment of a syringe and a plunger rod formed form shape-changeable package;

FIG. 12A is a perspective view of the plunger rod shown in FIG. 10 in an assembled configuration; and

FIG. 12B is a perspective view of the plunger rod shown in FIG. 11A in a disassembled configuration and ready for assembly.

To facilitate understanding, identical reference numerals have been used, where possible, to designate identical elements that are common to the figures. The figures are not drawn to scale.

### DETAILED DESCRIPTION

Before describing several exemplary embodiments of the disclosure, it is to be understood that the disclosure is not limited to the details of construction or process steps set forth in the following description. The disclosure is capable of other embodiments and of being practiced or being carried out in various ways.

The matters exemplified in this description are provided to assist in understanding exemplary embodiments of the disclosure. Before describing several exemplary embodiments of the disclosure, it is to be understood that the disclosure is not limited to the details of construction or process steps set forth in the following description. The disclosure is capable of other embodiments and of being practiced or being conducted in many ways.

With respect to terms used in this disclosure, the following definitions are provided. As used herein, the use of “a,” “an,” and “the” includes the singular and plural.

Reference to “medical device” refers to any device intended to be used for medical purposes, for example, a syringe. Reference to “syringe” includes syringes that are indicated for use with needles, nozzle, tubing, or for use in flush systems. As used herein, the term “syringe” refers to a simple pump-like device consisting of a plunger rod that fits in a barrel or tube. The plunger rod can be pulled or pushed along inside the barrel, allowing the syringe to take in and expel a liquid or gas through an opening at the open end of the barrel. The open end of the syringe may be fitted with a needle, nozzle, or tubing to help direct the flow of fluid into and out of the barrel. The syringe may be sterile or unsterile, depending upon the needs of the technician.

As used herein, the terms “package” or “packaging” includes any material used to wrap or protect a good or product, such as a syringe. As used herein-in “shape-changeable” refers to a package that can be changed in shape or size by one or more ways, including, but not limited to, collapsing, rolling, re-arranging, folding and the like.

Aspects of the syringe and syringe package system described herein facilitate disposal of packaging and syringe waste. The syringe and syringe package system disclosed herein is also.

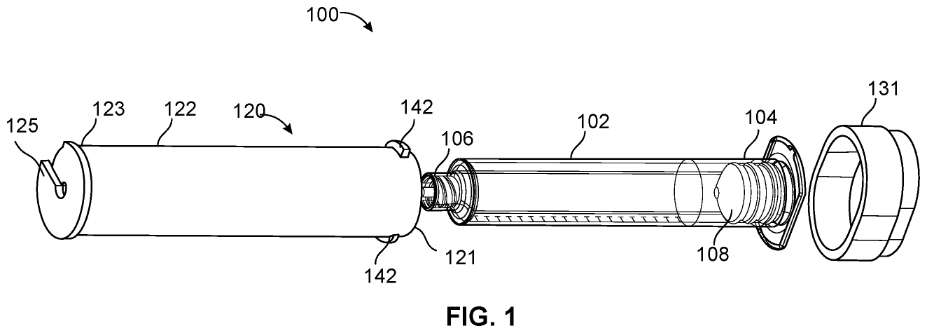

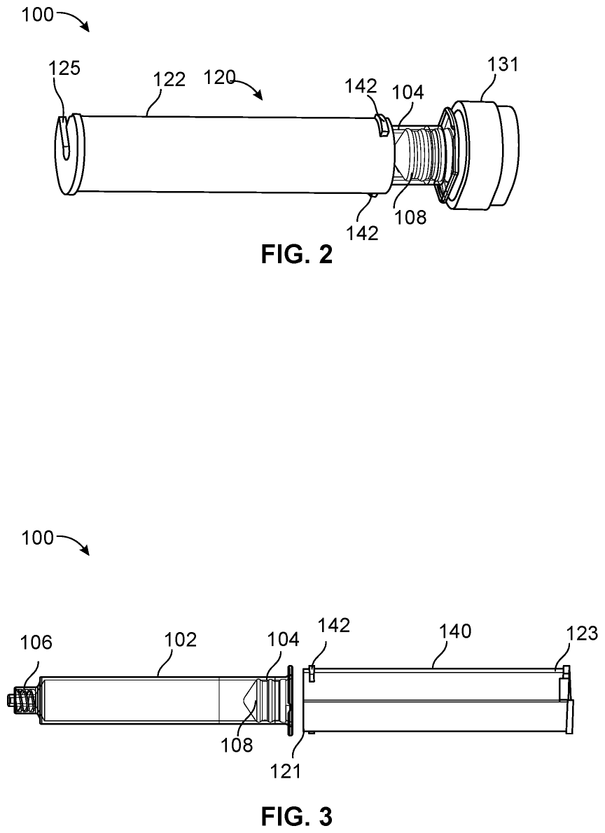

The following non-limiting description demonstrates principles according to one or more embodiments of the disclosure. Referring now to the drawings, a first aspect of the present disclosure is shown in FIGS. 1-6, showing a first embodiment of a syringe and syringe package system 100 comprising a syringe barrel 102 having an open proximal end 104 and a distal end 106. The distal end 106 of the syringe barrel 102 may be configured for connection to a medical device, a catheter, or a needle (not shown).

The syringe and syringe package system 100 further comprises a syringe stopper 108 placed in the open proximal end 104 of the syringe barrel 102. In addition, the syringe and syringe package system 100 further comprises a shape-changeable package 120, and the syringe barrel 102 and the syringe stopper 108 are contained within the shape-changeable package 120. The shape-changeable package 120 comprises a web of material 122 sized and arranged to contain the syringe barrel 102 and the syringe stopper 108 and configured to be converted to a plunger rod 140 that engages the syringe stopper 108 and is translatable within the open proximal end 104 of the syringe barrel 102. The plunger rod 140 according to one or more embodiments engages the stopper via lugs 142 extending from the web of material 122, which will be described further below.

In one or more embodiments of the syringe and syringe package system 100, the web of material 122 comprises a distal end 121 and a proximal end 123. As shown in the drawings, the lugs 142 extend radially outward from the distal end 121 from the web of material.

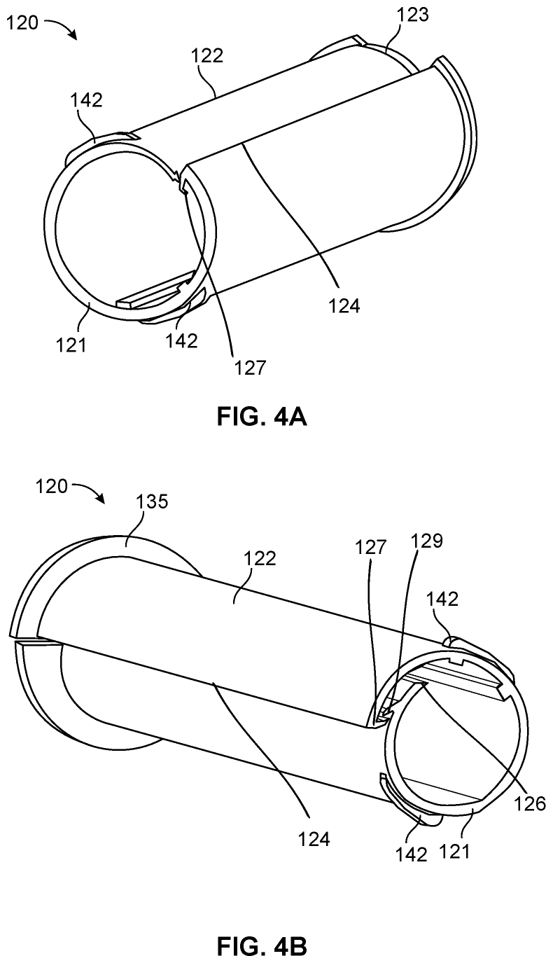

As best shown in FIGS. 4A and 4B, the web of material 122 further includes a first edge 124 extending from the distal end 121 to the proximal end 123, the first edge 124 comprising a crease 127, and a second edge 126 extending from the distal end 121 to the proximal end 123 and opposite the first edge 124. The second edge 126 comprises a locking element 129 configured to engage the crease 127. In one or more embodiments, the web of material 122 is in the form of a sheet of material that can be bent to form the crease 127 and the locking element 129 can be a projection or crimp extend outwardly from the web of material 122. The crease 127 can be any appropriately sized groove to allow the locking element 129 to fit therein. The locking element 129 is pushed into the crease 127 forming the web of material into a tubular shell that in a first configuration is sized to contain the syringe barrel 102 and the syringe stopper 108, forming a shape-changeable package 120 that maintains the syringe barrel 102 and the syringe stopper 108 in a clean and sterile condition until the syringe barrel and the syringe stopper are ready to be removed from the shape-changeable package 120. The shape-changeable package 120 further includes a distal end cap 125 and a proximal end cap 131.

Thus, the syringe and syringe package system 100 in the embodiments shown includes the locking element 129 comprising a radial projection extending outwardly from the web of material 122 and configured to releasably engage the crease 127. This arrangement facilitates conversion of the shape-changeable package 120 to the plunger rod 140.

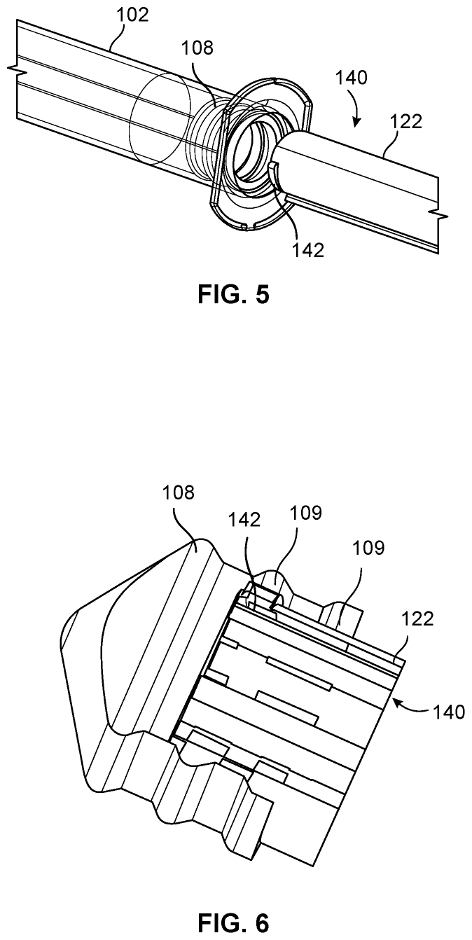

In the syringe and syringe package system 100, the shape-changeable package 120 is configured to be collapsible from a first rolled configuration having a first diameter to a second rolled configuration having a second diameter smaller than the first diameter. This first rolled configuration is shown in FIGS. 1 through 4A-B. The first diameter is sized so that the cavity formed by the web of material 122 can receive the syringe barrel 102 and the syringe plunger. The second diameter is sized so that when in the second rolled configuration shown in FIGS. 5 and 6, the lugs 142 engage the receiving grooves 109 in the syringe stopper 108. The receiving grooves 109 can be in the form of threads and the lugs 142 can be angled so that the plunger rod 140, which has been converted to the plunger rod 140 from the shape-changeable package 120 by rolling the web of material 122 to decrease the diameter of the rolled web of material 122 can be threaded into the syringe stopper. In other embodiments, the lugs 142 can be press fit into the grooves 109 in the syringe stopper 108.

Thus in one or more embodiments, the first diameter is sized and arranged to contain the syringe barrel 102 and the syringe stopper 108, and the second diameter is sized so that the distal end 121 of the plunger rod 140 fits within the syringe stopper 108 and the syringe barrel 102.

In the embodiment show, the syringe and syringe package system 100 further comprises a flange 135 extending from the proximal end, the flange providing thumb press for the plunger rod 140. In one or more embodiments, the web of material 122 comprises a biodegradable material. As used herein according to one or more embodiments, biodegradable refers to a material which is decomposed living organisms such as bacteria, and the decomposed material is innocuous. Therefore, a biodegradable material avoids pollution and is eco-friendly.

In one or more embodiments, the biodegradable material is soluble in a solvent such as alcohol. In specific embodiments, the biodegradable material comprises, consists essentially of or consists of zein. Zein is a protein polymer obtained from corn by extracting the polymer from corn. By replacing the packaging material and the plunger rod with the currently used plastic materials used for packaging and plunger rods, such as polypropylene material, landfill waste is greatly reduced. Plastic materials such as polypropylene are not biodegradable and not environmentally sustainable. According to one or more embodiments, the biodegradable material that is used for the web of material should have adequate tensile and compressive strength to withstand the forces applied to a plunger rod used for drawing fluid into syringes and expelling flued from syringes. It was determined that a web material made from zein could be used as a plunger rod according to the embodiments described herein.

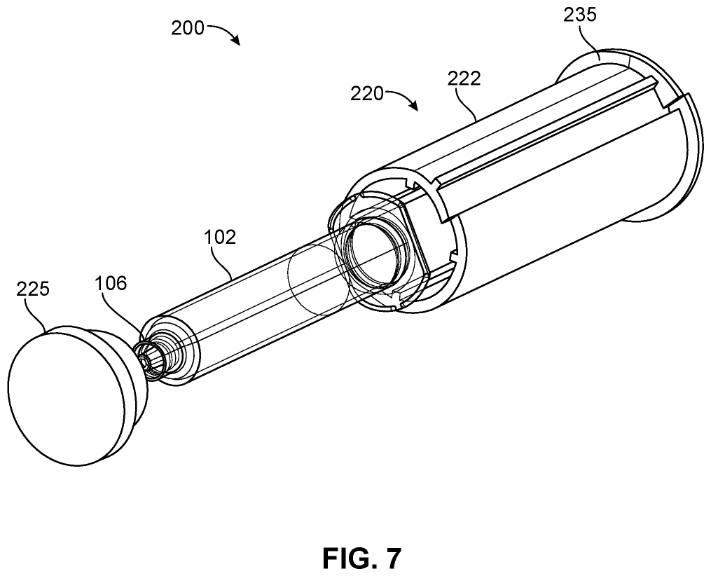

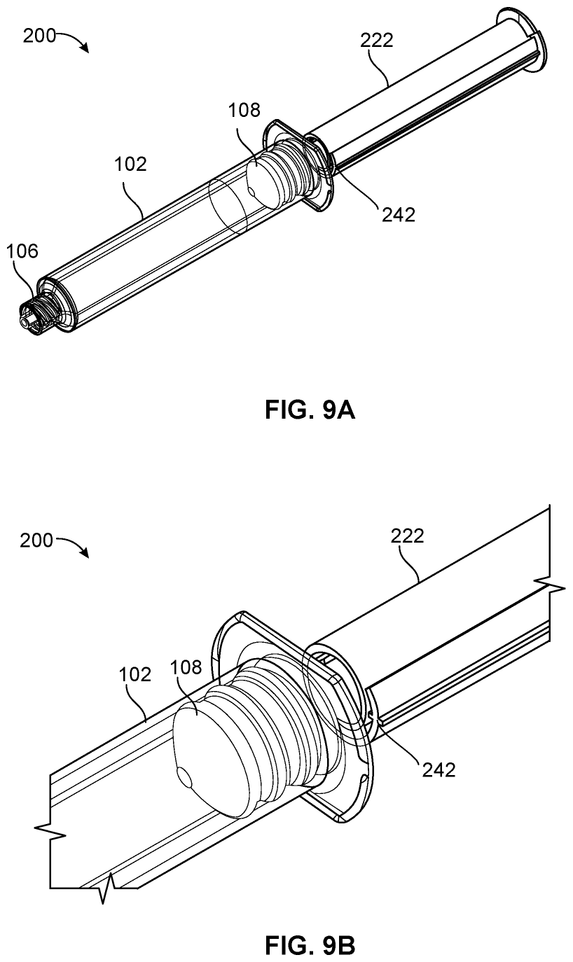

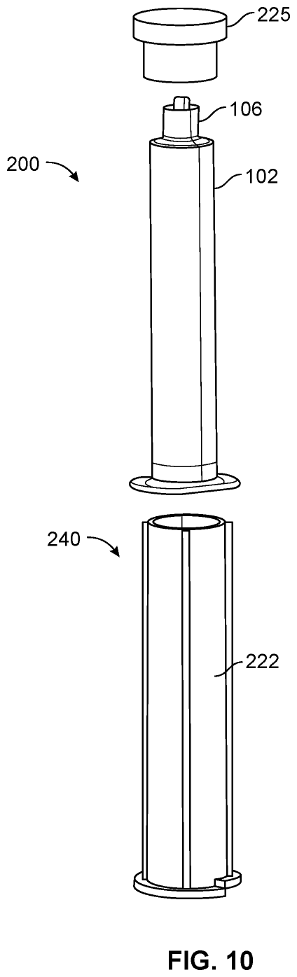

Referring now to FIGS. 7 through 10, a second embodiment a syringe and syringe package system 200 comprising a syringe barrel 102 having an open proximal end 104 and a distal end 106, similar to the first embodiment. The distal end 106 of the syringe barrel 102 is configured for connection to a medical device, a catheter, or a needle (not shown). Similar to the first embodiment, the syringe and syringe package system 200 further comprises a syringe stopper 108 placed in the open proximal end 104 of the syringe barrel 102. The shape-changeable package 220 further includes a distal end cap 225 and a proximal end cap 231.

In the second embodiment, a shape-changeable package 220 has a different configuration from the shape-changeable package 120 shown in the first embodiment. Only the different components are described herein. The syringe barrel 102 and the syringe stopper 108 are contained within the shape-changeable package 220. The shape-changeable package 220 comprises a web of material 222 sized and arranged to contain the syringe barrel 102 and the syringe stopper 108 and configured to be converted to a plunger rod 240 that engages the syringe stopper 108 and is translatable within the open proximal end 104 of the syringe barrel 102. The plunger rod 240 according to one or more embodiments engages the stopper via a keying surface 242 extending from the web of material 122, which will be described further below.

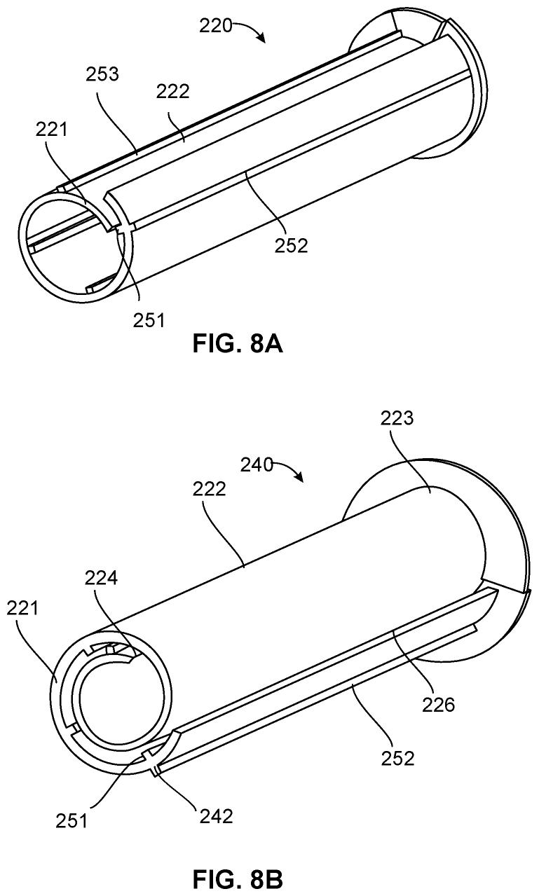

In the second embodiment of the syringe and syringe package system 200, the web of material 222 comprises a distal end 221 and a proximal end 223. The web of material 222 further includes a first edge 224 and a second edge 226 extending from the distal end 221 to the proximal end 223 and opposite the first edge 224. There is a first elongate projection 251 extending radially inwardly adjacent the second edge 226 and configured to engage the first edge 224 when the web of material is in a first rolled configuration shown in FIG. 8A sized and shaped to contain the syringe barrel 102 and the syringe stopper 108. In the configuration shown in FIG. 8A, the web of material 222 is configured as a shape-changeable package 220.

The shape-changeable package 220 in FIG. 7 and FIG. 8A is configured to be collapsible from the first rolled configuration having a first diameter to a second rolled configuration having a second diameter smaller than the first diameter. In the second embodiment, the web of material 222 further comprises a second elongate projection 252 adjacent second edge 226 and extending outwardly and a third elongate projection 253 adjacent the first edge 224 and extending radially outwardly. The first edge 224 and the first elongate projection 253 are positioned to engage each other when the shape-changeable package is in the first rolled configuration. In the second embodiment, the second elongate projection 252 provides a keying surface 242 at the distal end 221 to engage the syringe stopper 108. In the second embodiment, the syringe web of material further includes a flange 235, which functions as a thumb press when the shape-changeable package 220 is converted into a plunger rod 240 upon collapsing the shape-changeable package.

In the second embodiment, the biodegradable material is soluble in a solvent such as alcohol. In specific embodiments, the biodegradable material comprises, consists essentially of or consists of zein. Zein is a protein polymer obtained from corn by extracting the polymer from corn. By replacing the packaging material and the plunger rod with the currently used plastic materials used for packaging and plunger rods, such as polypropylene material, landfill waste is greatly reduced. Plastic materials such as polypropylene are not biodegradable and not environmentally sustainable. According to one or more embodiments, the biodegradable material that is used for the web of material should have adequate tensile and compressive strength to withstand the forces applied to a plunger rod used for drawing fluid into syringes and expelling flued from syringes. It was determined that a web material made from zein could be used as a plunger rod according to the embodiments described herein.

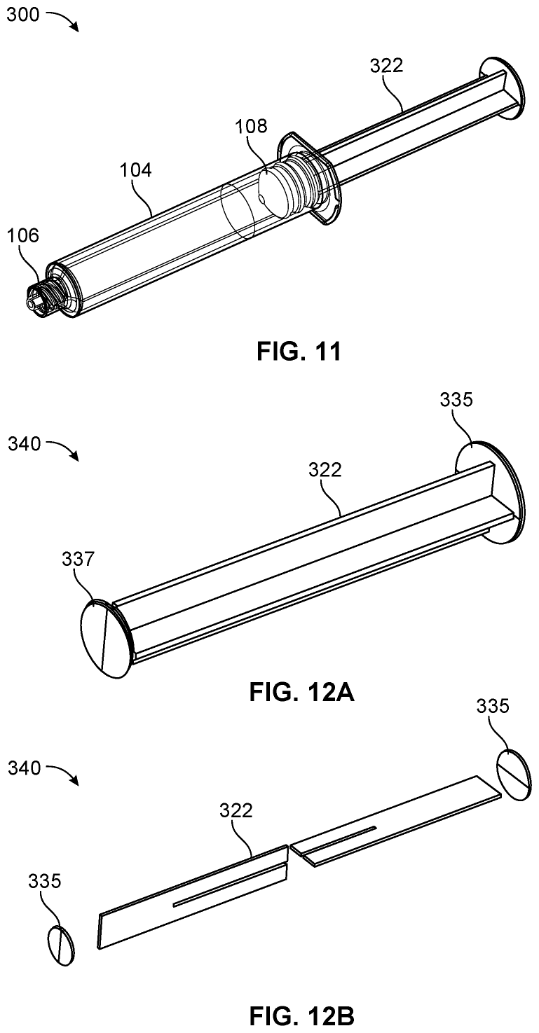

In a third embodiment of syringe and syringe package system 300 shown in FIGS. 11 and 12A-B, the web of material 322 used for the package is configured to be assembled into a t-shaped plunger rod 340 as shown. The web of material 322 in this embodiment is provided in a configuration where portions of the web of material 322 made from biodegradable material such as zein, include fold lines and/or perforations and slots formed in the web of material. Instructions are provided in the package indicating assembly of the web of material into a t-shaped plunger rod having a thumb press 335 and a stopper-engaging portion 337.

It will be appreciated that the syringe and syringe package system addresses a compelling need to reduce plastic waste generated by syringes and packaging. The proposed syringe and syringe package system will aid in reducing the carbon footprint of medical device disposal. The disclosure utilizes a biodegradable packaging material, one example being zein. Zein is one of a class of alcohol soluble proteins rich in prolamin, and generally recognized as safe for use in the coating of pharmaceuticals.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein has been described with reference to particular embodiments, it is to be understood that these embodiments are merely illustrative of the principles and applications of the present disclosure. It will be apparent to those skilled in the art that various modifications and variations can be made to the method and apparatus of the present disclosure without departing from the spirit and scope of the disclosure. Thus, it is intended that the present disclosure include modifications and variations that are within the scope of the appended claims and their equivalents.

What is claimed is:

1. A syringe and syringe package system comprising:
a syringe barrel having an open proximal end and a distal end;
a syringe stopper placed in the open proximal end of the syringe barrel; and
a shape-changeable package, the syringe barrel and the syringe stopper contained within the shape-changeable package, wherein the shape-changeable package comprises a web of material sized and arranged to contain the syringe barrel and the syringe stopper and configured to be converted to a plunger rod that engages the syringe stopper and is translatable within the open proximal end of the syringe barrel.

2. The syringe and syringe package system of claim 1, the web of material comprising a distal end, a proximal end, a first edge extending from the distal end to the proximal end, the first edge comprising a crease, and a second edge extending from the distal end to the proximal end and opposite the first edge, the second edge comprising a locking element configured to engage the crease.

3. The syringe and syringe package system of claim 2, wherein the shape-changeable package is configured to be collapsible from a first rolled configuration having a first diameter to a second rolled configuration having a second diameter smaller than the first diameter.

4. The syringe and syringe package system of claim 3, wherein the first diameter is sized and arranged to contain the syringe barrel and the second diameter is sized to fit within the syringe stopper and the syringe barrel.

5. The syringe and syringe package system of claim 4, wherein the locking element comprises a radial projection extending outwardly from the web of material and configured to releasably engage the crease.

6. The syringe and syringe package system of claim 2, further comprising lugs extending radially outwardly from the distal end and configured to engage the syringe stopper.

7. The syringe and syringe package system of claim 6, the syringe stopper comprising threads configured to engage the lugs.

8. The syringe and syringe package system of claim 6, further comprising a flange extending from the proximal end, the flange providing thumb press for the plunger rod.

9. The syringe and syringe package system of claim 2, wherein the web of material comprises biodegradable material.

10. The syringe and syringe package system of claim 9, wherein the biodegradable material comprises zein.

11. The syringe and syringe package system of claim 1, the web of material comprising a distal end, a proximal end, a first edge extending from the distal end to the proximal end, and a second edge extending from the distal end to the proximal end and opposite the first edge, and a first elongate projection extending radially inward adjacent the second edge and configured to engage the first edge when the web of material is in a first rolled configuration sized and shaped to contain the syringe barrel and the syringe stopper.

12. The syringe and syringe package system of claim 11, wherein the shape-changeable package is configured to be collapsible from the first rolled configuration having a first diameter to a second rolled configuration having a second diameter smaller than the first diameter.

13. The syringe and syringe package system of claim 12, the web of material further comprising a second elongate projection adjacent second edge and extending outwardly and a third elongate projection adjacent the first edge and extending outwardly, wherein the first elongate projection and the first are positioned to engage each other when the shape-changeable package is in the first rolled configuration.

14. The syringe and syringe package system of claim 13, wherein the second elongate projection provides a keying surface configured to engage the syringe stopper.

15. The syringe and syringe package system of claim 14, further comprising a flange extending from the proximal end, the flange providing thumb press for the plunger rod.

16. The syringe and syringe package system of claim 15, wherein the web of material comprises biodegradable material.

17. The syringe and syringe package system of claim 16, wherein the biodegradable material comprises zein.

18. The syringe and syringe package system of claim 1, wherein the web of material is configured to be assembled into a t-shaped plunger rod.

19. A syringe and syringe package system comprising:
a syringe barrel having an open proximal end and a distal end;
a syringe stopper placed in the open proximal end of the syringe barrel; and
a shape-changeable package, the syringe barrel and the syringe stopper contained within the shape-changeable package, wherein the shape-changeable package comprises a rolled web of biodegradable material sized and arranged to contain the syringe barrel and the syringe stopper and configured to be converted to a plunger rod that engages the syringe stopper and is translatable within the open proximal end of the syringe barrel.

20. The syringe and syringe package system of claim 19, wherein the biodegradable material comprises zein.
